SEQUENCE LISTING (14 sequences)

SEQ ID NO: 1 - protein, 24 aa, Homo sapiens
MPMGSLQPLATLYLLGMLVASCLG

SEQ ID NO: 2 - protein, 522 aa, Homo sapiens
MDFLLLQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLAL

SEQ ID NO: 3 - protein, 142 aa, Homo sapiens
MGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAEKVRRAIEQLAAMD

SEQ ID NO: 4 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 5 - protein, 314 aa, Homo sapiens
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 6 - protein, 509 aa, Homo sapiens
MERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 7 - protein, 1667 aa, synthetic construct
LDFLLLQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLALMGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAEKVRRAIEQLAAMDMQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRRMPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEMERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 8 - protein, 1691 aa, synthetic construct
MPMGSLQPLATLYLLGMLVASCLGLDFLLLQDPASTCVPEPASQHTLRSGPGCLQQPEQQGVRDPGGIWAKLGAAEASAERLQGRRSRGASGSEPQQMGSDVRDLNALLPAVPSLGGGGGCALPVSGAAQWAPVLDFAPPGASAYGSLGGPAPPPAPPPPPPPPPHSFIKQEPSWGGAEPHEEQCLSAFTVHFSGQFTGTAGACRYGPFGPPPPSQASSGQARMFPNAPYLPSCLESQPAIRNQGYSTVTFDGTPSYGHTPSHHAAQFPNHSFKHEDPMGQQGSLGEQQYSVPPPVYGCHTPTDSCTGSQALLLRTPYSSDNLYQMTSQLECMTWNQMNLGATLKGVAAGSSSSVKWTEGQSNHSTGYESDNHTTPILCGAQYRIHTHGVFRGIQDVRRVPGVAPTLVRSASETSEKRPFMCAYPGCNKRYFKLSHLQMHSRKHTGEKPYQCDFKDCERRFSRSDQLKRHQRRHTGVKPFQCKTCQRKFSRSDHLKTHTRTHTGKTSEKPFSCRWPSCQKKFARSDELVRHHNMHQRNMTKLQLALMGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAEKVRRAIEQLAAMDMQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRRMPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEMERRRLWGSIQSRYISMSVWTSPRRLVELAGQSLLKDEALAIAALELLPRELFPPLFMAAFDGRHSQTLKAMVQAWPFTCLPLGVLMKGQHLHLETFKAVLDGLDVLLAQEVRPRRWKLQVLDLRKNSHQDFWTVWSGNRASLYSFPEPEAAQPMTKKRKVDGLSTEAEQPFIPVEVLVDLFLKEGACDELFSYLIEKVKRKKNVLRLCCKKLKIFAMPMQDIKMILKMVQLDSIEDLEVTCTWKLPTLAKFSPYLGQMINLRRLLLSHIHASSYISPEKEEQYIAQFTSQFLSLQCLQALYVDSLFFLRGRLDQLLRHVMNPLETLSITNCRLSEGDVMHLSQSPSVSQLSVLSLSGVMLTDVSPEPLQALLERASATLQDLVFDECGITDDQLLALLPSLSHCSQLTTLSFYGNSISISALQSLLQHLIGLSNLTHVLYPVPLESYEDIHGTLHLERLAYLHARLRELLCELGRPSMVWLSANPCPHCGDRTFYDPEPILCPCFMPN

SEQ ID NO: 9 - protein, 799 aa, synthetic construct
GSVETPRGLIKVTSYDGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGAAATGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARQFKFSRVCKPKSSLEETEVLFVFIGYDRKARTHNSYKLSSTLTNIYTGSRLHEAGC

SEQ ID NO: 10 - protein, 799 aa, synthetic construct
GSVETPRGLIKVTSYAGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGAAATGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARQFKFSRVCKPKSSLEETEVLFVFIGYDRKARTHNPYKLSSTLTNIYTGSRLHEAGC

SEQ ID NO: 11 - protein, 799 aa, synthetic construct
GSVETPRGLIKVTSYAGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKRMRTTNPKETKIEIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGAAATGTLRNYDPRINLVPVNRRLPHALVLHHNEHPQSDFSSFVSKLKGRTVLVVGEKLSVPGKKVDWLSDQPEATFRARLDLGIPGDVPKYDIVFINVRTPYKYHHYQQCEDHAIKLSMLTKKACLHLNPGGTCVSIGYGYADRASESIIGAIARQFKFSRVCKPKSSHEETEVLFVFIGYDRKARTHNPYKLSSTLTNIYTGSRLHEAGC

SEQ ID NO: 12 - protein, 799 aa, synthetic construct
  VARIANT 16: note = X = A or D
  VARIANT 328: note = X = K or R
  VARIANT 340: note = X = V or E
  VARIANT 652: note = X = M or K
  VARIANT 659: note = X = R or Q
  VARIANT 682: note = X = I or V
  VARIANT 684: note = X = V or I
  VARIANT 757: note = X = L or H
  VARIANT 778: note = X = P or S
GSVETPRGLIKVTSYXGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKXMRTTNPKETKIXIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGAAATGTLRNYDPRINLVPVNRRLPHALVLHHNEHPQSDFSSFVSKLKGRTVLVVGEKLSVPGKXVDWLSDXPEATFRARLDLGIPGDVPKYDIXFXNVRTPYKYHHYQQCEDHAIKLSMLTKKACLHLNPGGTCVSIGYGYADRASESIIGAIARQFKFSRVCKPKSSXEETEVLFVFIGYDRKARTHNXYKLSSTLTNIYTGSRLHEAGC

SEQ ID NO: 13 - protein, 794 aa, Venezuelan equine encephalitis virus
GSVETPRGLIKVTSYAGEDKIGSYAVLSPQAVLKSEKLSCIHPLAEQVIVITHSGRKGRYAVEPYHGKVVVPEGHAIPVQDFQALSESATIVYNEREFVNRYLHHIATHGGALNTDEEYYKTVKPSEHDGEYLYDIDRKQCVKKELVTGLGLTGELVDPPFHEFAYESLRTRPAAPYQVPTIGVYGVPGSGKSGIIKSAVTKKDLVVSAKKENCAEIIRDVKKMKGLDVNARTVDSVLLNGCKHPVETLYIDEAFACHAGTLRALIAIIRPKKAVLCGDPKQCGFFNMMCLKVHFNHEICTQVFHKSISRRCTKSVTSVVSTLFYDKKMRTTNPKETKIVIDTTGSTKPKQDDLILTCFRGWVKQLQIDYKGNEIMTAAASQGLTRKGVYAVRYKVNENPLYAPTSEHVNVLLTRTEDRIVWKTLAGDPWIKTLTAKYPGNFTATIEEWQAEHDAIMRHILERPDPTDVFQNKANVCWAKALVPVLKTAGIDMTTEQWNTVDYFETDKAHSAEIVLNQLCVRFFGLDLDSGLFSAPTVPLSIRNNHWDNSPSPNMYGLNKEVVRQLSRRYPQLPRAVATGRVYDMNTGTLRNYDPRINLVPVNRRLPHALVLHHNEHPQSDFSSFVSKLKGRTVLVVGEKLSVPGKMVDWLSDRPEATFRARLDLGIPGDVPKYDIIFVNVRTPYKYHHYQQCEDHAIKLSMLTKKACLHLNPGGTCVSIGYGYADRASESIIGAIARQFKFSRVCKPKSSLEETEVLFVFIGYDRKARTHNPYKLSSTLTNIYTGSRLHEAGC

SEQ ID NO: 14 - protein, 5 aa, synthetic construct
TGAAA

(19) United States
(12) Patent Application Publication
(10) Pub. No.: US 2024/0350605 A1
(43) Pub. Date: Oct. 24, 2024

(54) TEMPERATURE-CONTROLLABLE, RNA IMMUNOTHERAPEUTIC FOR CANCER

(71) Applicant: Elixirgen Therapeutics, Inc., Baltimore, MD (US)

(72) Inventor: Minoru S.H. KO, Cockeysville, MD (US)

(73) Assignee: Elixirgen Therapeutics, Inc., Baltimore, MD (US)

(21) Appl. No.: 18/688,336

(22) PCT Filed: Aug. 31, 2022
(86) PCT No.: PCT/US2022/075789

§ 371 Date: Feb. 29, 2024

Related U.S. Application Data
(60) Provisional application No. 63/390,216, filed on Jul. 18, 2022.
(60) Provisional application No. 63/341,318, filed on May 12, 2022.
(60) Provisional application No. 63/240,280, filed on Sep. 2, 2021.

(51) Int. Cl. A61K 39/00; A61K 9/00; A61K 47/36; A61P 35/00; C07K 14/005; C07K 14/47; C12N 15/86

(52) U.S. Cl. CPC A61K 39/001189; A61K 9/0021; A61K 39/00115; A61K 39/001153; A61K 39/001186; A61K 39/001188; A61K 47/36; A61P 35/00; C07K 14/005; C07K 14/4748; C12N 15/86; A61K 2039/53; A61K 2039/54; A61K 2039/572; A61K 2039/575; A61K 2039/70; C07K 2319/02; C12N 2770/36122; C12N 2770/36134; C12N 2770/36143; C12N 2810/855

(57) ABSTRACT

The present disclosure relates to mRNA, self-replicating RNA, and temperature-sensitive, self-replicating RNA encoding a cancer antigen. The RNA constructs are suitable for cancer immunotherapy in a mammalian subject, such as a human subject.

### CROSS-REFERENCE TO RELATED APPLICATION(S)

This application claims the benefit of U.S. Provisional Application No. 63/390,216, filed Jul. 18, 2022, U.S. Provisional Application No. 63/341,318, filed May 12, 2022, and U.S. Provisional Application No. 63/240,280, filed Sep. 2, 2021, each of which is hereby incorporated by reference in its entirety.

### SUBMISSION OF ELECTRONIC SEQUENCE LISTING

The content of the of the electronic sequence listing (699442001540SEQLIST.xml; Size: 25,374 bytes; and Date of Creation: Aug. 30, 2022) is herein incorporated by reference in its entirety.

### FIELD

The present disclosure relates to mRNA, self-replicating RNA, and temperature-sensitive, self-replicating RNA encoding a cancer antigen. The RNA constructs are suitable for cancer immunotherapy in a mammalian subject, such as a human subject.

### BACKGROUND

Immunotherapy can be effective in treating cancer and has become more widely used. One therapeutic strategy is to inject immunogenic compositions including antigens that are expressed in tumor cells into cancer patients. Tumor-associated antigens (TAA) are expressed in tumor cells, but are also expressed in embryonic cells or expressed at low levels in normal cells. Tumor-specific antigens (TSA), also called neoantigens, are expressed only in tumor cells, and are often expressed from genes that are mutated in tumor cells. Cancer immunotherapy relies on the induction of a cytotoxic T lymphocyte (CTL) response against cancer cells.

There is a need in the art for cancer immunotherapies that induce potent TAA- or TSA-specific cellular immune responses to destroy tumor cells that express a TAA or a TSA.

### BRIEF SUMMARY

The present disclosure related to the use of a cancer antigen (TAA and/or TSA) to induce a cellular immune response against cancer cells. In some embodiments, a temperature-controllable, self-replicating RNA vaccine platform is utilized. In an exemplary embodiment, the WT1 protein is expressed in host cells from a temperature-controllable, self-replicating RNA (c-srRNA) to induce a potent cellular immune response against WT1-expressing tumor cells. A c-srRNA is also referred to herein as a temperature-sensitive self-replicating RNA (srRNAts). Importantly, the c-srRNA-WT1 immunotherapeutic (EXG-5101) was found to inhibit tumor growth and even reduce size of established tumors in a preclinical model. Thus, the c-srRNA platform described herein is a suitable vector for expression of a tumor-associated antigen (TAA) such as WT1, NY-ESO-1, MAGEA3, BIRC5 (also known as SURVIVIN), PRAME or a tumor-specific antigen (TSA), also known as a neoantigen. In some embodiments, the c-srRNA is used to express a fusion protein of two or more TAAs, TSAs, or a combination of a TAA and a TSA.

Among other embodiments, the present disclosure provides compositions comprising an excipient and a temperature-controllable, self-replicating RNA (c-srRNA). In some embodiments, the composition comprises a chitosan. In some embodiments, the chitosan is a low molecular weight (about 3-5 kDa) chitosan oligosaccharide, such as chitosan oligosaccharide lactate. In some embodiments, the composition does not comprise liposomes or lipid nanoparticles.

### BRIEF DESCRIPTION OF THE DRAWINGS

FIG. 1 shows a schematic diagram of the mechanism for induction of cellular (CD4+ and CD8+ T cell) immune responses after intradermal injection of temperature-controllable, self-replicating RNA (referred to herein as “c-srRNA” or “srRNAts”).

FIG. 2 shows a schematic diagram of cancer antigen expressed from a temperature-controllable self-replicating RNA (c-srRNA). In an exemplary embodiment, the coding region of a human Wilms tumor (WT1) protein is the gene of interest (GOI) inserted within the c-srRNA. The EXG-5101 antigen is a fusion protein comprising the signal peptide sequence from the human CD5 antigen (CD5-SP) set forth as SEQ ID NO:1, and the amino acid sequence of the human WT1 protein set forth as SEQ ID NO:1 (Isoform D, GenBank No. NM_024426.6, NCBI No. NP_077744.4). The coding sequence of WT1 Isoform D has a non-AUG (CUG) translation initiation codon.

FIG. 3 shows a schematic diagram of an exemplary method for stimulating an immune response against a cancer antigen in a human subject. c-srRNA is functional at a permissive temperature (e.g., 30-35° C.), but non-functional at a non-permissive temperature (e.g., >37° C.). The temperature at or just below the surface of a human body (surface body temperature), which is around 31-34° C., is lower than the core body temperature of the human body, which is around 37° C. The c-srRNA is directly delivered by intradermal and subcutaneous administration to cells of a subject that are at the permissive, surface body temperature.

FIG. 4 illustrates the testing of the EXG-5101 mRNA vaccine in a syngeneic mouse tumor model.

FIG. 5 shows graphs of the growth of tumors in BALB/c mice injected with a placebo (PBO), 5 μg, or 25 μg of the EXG-5101 mRNA vaccine. The average and standard deviation (error bars) of five mice (n=5) are shown for each group. By Day 7 post-tumor inoculation, all three groups of mice developed tumors. However, by Day 25 post-tumor inoculation (Day 18 post-injection), tumor growth was retarded in mice injected with the EXG-5101 mRNA vaccine in a dose-dependent manner. In contrast, tumors continued to grow in mice injected with the placebo.

FIG. 6A-B shows the induction of a tumor-associated antigen-reactive cellular immune response by intradermal injection of EXG-5101 mRNA (temperature-controllable, self-replicating RNA encoding human WT1 gene). FIG. 6A illustrates the experimental procedure. FIG. 6B shows the results of ELISpot assays of splenocytes obtained from five (n=5) mice each that had been immunized by intradermal injection of 25 μg of EXG-5101 or a placebo (buffer only). The left panel shows the frequency of interferon-gamma (IFN-γ) spot-forming cells (SFC) per 1×10\hat{ }6 splenocytes that were stimulated by a pool of 110 peptides that covers the human WT1 protein (15 mers with 11 amino acid overlaps: JPT Peptide Technologies, Catalog #PM-WT1). The right panel shows the frequency of interleukin-4 (IL-4) SFC per 1×10\hat{ }6 splenocytes that were stimulated by a pool of 110 peptides that covers the human WT1 protein (15 mers with 11 amino acid overlaps: JPT Peptide Technologies, Catalog #PM-WT1) The average and standard deviation (error bars) are shown for each group.

FIG. 7 shows a schematic diagram of a fusion protein comprising multiple tumor-associated antigens expressed from a temperature-controllable self-replicating RNA (c-srRNA). In an exemplary embodiment, the EXG-5105 antigen is a fusion protein comprising the signal peptide sequence from the human CDS antigen (CD5-SP) set forth as SEQ NO: 1; the amino acid sequence of the human WT1 protein set forth as SEQ ID NO:2 [Isoform D, GenBank No. NM_024426.6, NCBI No. NP_077744.4: the coding sequence of WT1 Isoform D has a non-AUG (CUG) translation initiation codon]; the amino acid sequence of the human BIRC5 (also known as SURVIVIN) protein set forth as SEQ ID NO:3 (GenBank No. NM_001168); the amino acid sequence of the human NY-ESO-1 protein set forth as SEQ NO: 4 (GenBank No. NM_001327); the amino acid sequence of the human MAGEA3 protein set forth as SEQ NO: 5 (GenBank No. NM_005362); and the amino acid sequence of the human PRAME protein set forth as SEQ ID NO:6 (GenBank No. NM_001291715). The amino acid sequence of the TAA fusion protein is set forth as SEQ ID NO:7, and the amino acid sequence of the CD5-SP plus the TAA fusion protein is set forth as SEQ ID NO:8

FIG. 8A-F shows the induction of a tumor-associated antigen-reactive cellular immune response by intradermal injection of EXG-5105 mRNA (temperature-controllable, self-replicating RNA encoding the fusion protein of human WT1 gene, human BIRC5 (SURVIVIN), human NY-ESO-1, human MAGEA3, and human PRAME. FIG. 8A illustrates the experimental procedure. On day 0, a total of 10 BALB/c female mice were used for the experiment; five mice received the intradermal injection of 25 μg each of EXG-5105, and five mice received the intradermal injection of a placebo (buffer only). On day 14, splenocytes were collected from each mouse and were tested by the ELISpot assays for immune response against WT1 and NY-ESO-1 as exemplary antigens coded on EXG-5105 mRNA vaccine. FIG. 8B shows the frequency of cytokine (left, interferon-gamma [IFN-γ]; right, Interleukin-4 [IL-4)]) spot-forming cells (SFC) per 1×10\hat{ }6 splenocytes that were stimulated by a pool of 110 peptides that covers the human WT1 protein (15 mers with 11 amino acid overlaps: JPT Peptide Technologies, Catalog #PM-WT1). FIG. 8C shows the frequency of cytokine (left, interferon-gamma [IFN-γ]; right, Interleukin-4 [IL-4)]) spot-forming cells (SFC) per 1×10\hat{ }6 splenocytes that were stimulated by a pool of peptides that covers the human NY-ESO-1 protein (15 mers with 11 amino acid overlaps: Miltenyi Biotec, Catalog #130-095-380). FIG. 8D shows the frequency of cytokine (left, interferon-gamma [IFN-γ]; right, Interleukin-4 [IL-4)]) spot-forming cells (SFC) per 1×10\hat{ }6 splenocytes that were stimulated by a pool of peptides that covers the human MAGEA3 protein (15 mers with 11 amino acid overlaps: JPT PepMix MAGEA3, UniProt ID: P43357, Cat #PM-MAGEA3). FIG. 8E shows the frequency of cytokine (left, interferon-gamma [IFN-γ]; right, Interleukin-4 [IL-4)]) spot-forming cells (SFC) per 1×10\hat{ }6 splenocytes that were stimulated by a pool of peptides that covers the human BIRC5 (SURVIVIN) protein (15 mers with 11 amino acid overlaps: JPT PepMix Survivin-1, UniProt ID: 015392, Cat #PM-Survivin). FIG. 8F shows the frequency of cytokine (left, interferon-gamma [IFN-γ]; right, Interleukin-4 [IL-4)]) spot-forming cells (SFC) per 1×10\hat{ }6 splenocytes that were stimulated by a pool of peptides that covers the human PRAME protein (15 mers with 11 amino acid overlaps: JPT PepMix PRAME (OIP4), UniProt ID: P43357, Cat #PM-OIP4).

FIG. 9A-B shows a comparison of srRNA constructs for T-cell-inducibility. FIG. 9A illustrates the experimental procedures. On day 0, mice were intradermally injected with either placebo (PBO, buffer only), srRNAO, c-srRNA1, c-srRNA3, or c-srRNA4. The srRNA0, c-srRNA1, c-srRNA3, and c-srRNA4 encode the same RBD of SARS-COV-2. On day 14, mice were sacrificed and splenocytes were isolated for ELISpot assays against the RBD protein. FIG. 9B shows the number of IFN-γ spot-forming cells (SFC) in 1×10\hat{ }6 splenocytes from immunized mice restimulated by culturing in the splenocytes in the presence or absence of a pool of 53 peptides (15 mers with 11 amino acid overlaps) that covers the SARS-COV-2 RBD (original strain). The average and standard deviation (error bars) are shown for each group.

### DETAILED DESCRIPTION

Cancer immunotherapy is contemplated to be best achieved through immunogenic compositions that mainly rely on the induction of cellular immunity (i.e., T-cell-inducing vaccines involving CD8+ killer T cells and CD4+ helper T cells). The present disclosure provides mRNA, self-replicating RNA (srRNA), and temperature-controllable, self-replicating RNA (c-srRNA) encoding one or more cancer antigens such as Tumor-associated antigens (TAA) and Tumor-specific antigens (TSA, also called neoantigens). Thus, the present disclosure provides a cellular immunity-based platform for cancer immunotherapy. Wilms tumor 1 (WT1) is a tumor-associated antigen (TAA), which is expressed in a broad range of tumors, but is only expressed in embryonic tissues and very limited cell types in adults. Accordingly, in some embodiments the c-srRNA encodes WT1. In some embodiments, the c-srRNA encodes BIRC5 (aka SURVIVIN). In some embodiments, the c-srRNA encodes NY-ESO-1. In some embodiments, the c-srRNA encodes MAGEA3. In some embodiments, the c-srRNA encodes PRAME. In further embodiments, the c-srRNA encodes one, two, three, four or all five cancer antigens of the group consisting of WT1, BIRC5, NY-ESO-1, MAGEA3, and PRAME.

### Cellular Immunity-Based mRNA Immunotherapeutic Platform

The vaccine platform is described in part in Elixirgen's earlier patent application [PCT/US20/67506, now published as WO 2021/138447 A1]. This vaccine platform is optimized to induce cellular immunity, which becomes possible by combining existing knowledge of vaccine biology with temperature-controllable self-replicating mRNA (c-srRNA) based on an Alphavirus, such as the Venezuelan equine encephalitis virus (VEEV). The terms c-srRNA and srRNAts are used interchangeably throughout the present disclosure, with srRNA Its2 (described in WO 2021/138447 A1) being an exemplary embodiment. c-srRNA is based on srRNA, which is also known as self-amplifying mRNA (saRNA or SAM), by incorporating small amino acid changes in the Alphavirus replicase that provide temperature-sensitivity. Elixirgen's c-srRNA is functional at a permissive temperature range of about 30-35° C., but is not functional at a non-permissive temperature at or above about 37° C. It carries all the benefits of mRNA platforms: no genome integration, rapid development and deployment, and a simple GMP (good manufacturing process) process, as well as the additional advantages of srRNA platforms (i.e., a predecessor of our c-srRNA platform) compared to mRNA platforms, particularly longer expression [Johanning et al., 1995] and higher immunogenicity at a lower dosage [Brito et al., 2014]. However, this simple temperature-controllable feature makes it possible to pull together many desirable features of T-cell inducing vaccine as briefly described below.

In brief, srRNA1ts2 is a temperature-sensitive, self-replicating VEEV-based RNA replicon developed for transient expression of a heterologous protein. Temperature-sensitivity is conferred by an insertion of five amino acids residues within the non-structural Protein 2 (nsP2) of VEEV. The nsP2 protein is a helicase/proteinase, which along with nsP1, nsP3 and nsP4 constitutes a VEEV replicase. srRNA1ts2 does not contain VEEV structural proteins (capsid, E1, E2 and E3). The disclosure of WO 2021/138447 A1 of Elixirgen Therapeutics, Inc. is hereby incorporated by reference. In particular, Example 3, FIG. 12, and SEQ ID NOs. 29-49 of WO 2021/138447 A1 are hereby incorporated by reference.

### General Techniques and Definitions

The practice of the present disclosure will employ, unless otherwise indicated, conventional techniques of molecular biology (including recombinant techniques), microbiology, cell biology, biochemistry and immunology, which are within the skill of the art.

As used herein and in the appended claims, the singular forms “a”, “an”, and “the” include plural references unless indicated otherwise. For example, “an” excipient includes one or more excipients.

The phrase “comprising” as used herein is open-ended, indicating that such embodiments may include additional elements. In contrast, the phrase “consisting of” is closed, indicating that such embodiments do not include additional elements (except for trace impurities). The phrase “consisting essentially of” is partially closed, indicating that such embodiments may further comprise elements that do not materially change the basic characteristics of such embodiments.

The term “about” as used herein in reference to a value, encompasses from 90% to 110% of that value (e.g., molecular weight of about 5,000 daltons when used in reference to a chitosan oligosaccharide refers to 4,500 daltons to 5,500 daltons).

The term “antigen” refers to a substance that is recognized and bound specifically by an antibody or by a T cell antigen receptor. Antigens can include peptides, polypeptides, proteins, glycoproteins, polysaccharides, complex carbohydrates, sugars, gangliosides, lipids and phospholipids; portions thereof and combinations thereof. In the context of the present disclosure, the term “antigen” typically refers to a polypeptide or protein antigen at least eight amino acid residues in length, which may comprise one or more post-translational modifications.

The terms “polypeptide” and “protein” are used interchangeably to refer to a polymer of amino acid residues, and are not limited to a certain length unless otherwise specified. Polypeptides may include natural amino acid residues or a combination of natural and non-natural amino acid residues The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity (e.g., antigenicity).

The terms “isolated” and “purified” as used herein refers to a material that is removed from at least one component with which it is naturally associated (e.g., removed from its original environment). The term “isolated,” when used in reference to a recombinant protein, refers to a protein that has been removed from the culture medium of the host cell that produced the protein. In some embodiments, an isolated protein (e.g., WT1 protein) is at least 75%, 90%, 95%, 96%, 97%, 98% or 99% pure as determined by HPLC.

An “effective amount” or a “sufficient amount” of a substance is that amount sufficient to effect beneficial or desired results, including clinical results, and, as such, an “effective amount” depends upon the context in which it is being applied. In the context of administering a composition of the present disclosure comprising an mRNA encoding an antigen, an effective amount contains sufficient mRNA to stimulate an immune response (preferably a cellular immune response against the antigen).

In the present disclosure, the terms “individual” and “subject” refer to a mammals. “Mammals” include, but are not limited to, humans, non-human primates (e.g., monkeys), farm animals, sport animals, rodents (e.g., mice and rats) and pets (e.g., dogs and cats). In some preferred embodiments, the subject is a human subject.

The term “dose” as used herein in reference to a composition comprising a mRNA encoding an antigen refers to a measured portion of the taken by (administered to or received by) a subject at any one time. Administering a composition of the present disclosure to a subject in need thereof, comprises administering an effective amount of a composition comprising a mRNA encoding an antigen to stimulate an immune response to the antigen in the subject.

“Stimulation” of a response or parameter includes eliciting and/or enhancing that response or parameter when compared to otherwise same conditions except for a parameter of interest, or alternatively, as compared to another condition (e.g., increase in antigen-specific cytokine secretion after administration of a composition comprising or encoding the antigen as compared to administration of a control composition not comprising or encoding the antigen). For example, “stimulation” of an immune response (e.g., Th1 response) means an increase in the response. Depending upon the parameter measured, the increase may be from 2-fold to 200-fold or over, from 5-fold to 500-fold or over, from 10-fold to 1000-fold or over, or from 2, 5, 10, 50, or 100-fold to 200, 500, 1,000, 5,000, or 10,000-fold.

Conversely, “inhibition” of a response or parameter includes reducing and/or repressing that response or parameter when compared to otherwise same conditions except for a parameter of interest, or alternatively, as compared to another condition. For example, “inhibition” of an immune response (e.g., Th2 response) means a decrease in the response. Depending upon the parameter measured, the decrease may be from 2-fold to 200-fold, from 5-fold to 500-fold or over, from 10-fold to 1000-fold or over, or from 2, 5, 10, 50, or 100-fold to 200, 500, 1,000, 2,000, 5,000, or 10,000-fold.

The relative terms “higher” and “lower” refer to a measurable increase or decrease, respectively, in a response or parameter when compared to otherwise same conditions except for a parameter of interest, or alternatively, as compared to another condition. For instance, a “higher antibody titer” refers to an antigen-reactive antibody titer as a consequence of administration of a composition of the present disclosure comprising an mRNA encoding an antigen that is at least 2, 3, 4, 5, 6, 7, 8, 9, or 10-fold above an antigen-reactive antibody titer as a consequence of a control condition (e.g., administration of a comparator composition that does not comprise the mRNA or comprises a control mRNA that does not encode the antigen). Likewise, a “lower antibody titer” refers to an antigen-reactive antibody titer as a consequence of a control condition (e.g., administration of a comparator composition that does not comprise the mRNA or comprises a control mRNA that does not encode the antigen) that is at least 2, 3, 4, 5, 6, 7, 8, 9, or 10-fold below an antigen-reactive antibody titer as a consequence of administration of a composition of the present disclosure comprising an mRNA encoding an antigen.

As used herein the term “immunization” refers to a process that increases a mammalian subject's reaction to antigen and therefore improves its ability to resist or overcome infection and/or resist disease.

The term “vaccination” as used herein refers to the introduction of a vaccine into a body of a mammalian subject.

As used herein, “percent (%) amino acid sequence identity” and “percent identity” and “sequence identity” when used with respect to an amino acid sequence (reference polypeptide sequence) is defined as the percentage of amino acid residues in a candidate sequence (e.g., the subject antigen) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into an antigen of interest and the products screened for a desired activity, e.g., increased stability and/or immunogenicity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

Conservative amino acid substitutions will involve exchanging a member of one of these classes with another member of the same class. Non-conservative amino acid substitutions will involve exchanging a member of one of these classes with a member of another class.

As used herein, the term “excipient” refers to a compound present in a composition comprising an active ingredient (e.g., mRNA encoding an antigen). Pharmaceutically acceptable excipients are inert pharmaceutical compounds, and may include for instance, solvents, bulking agents, buffering agents, tonicity adjusting agents, and preservatives (Pramanick et al., Pharma Times, 45:65-77, 2013). In some embodiments the compositions of the present disclosure comprise an excipient that functions as one or more of a solvent, a bulking agent, a buffering agent, and a tonicity adjusting agent (e.g., sodium chloride in saline may serve as both an aqueous vehicle and a tonicity adjusting agent).

### Optimized for Intradermal Delivery for Cellular Immunity

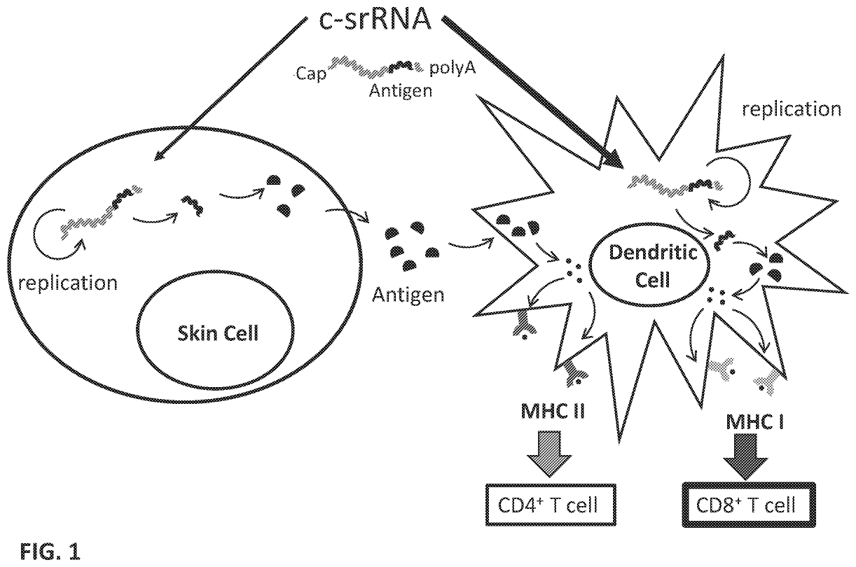

Intradermal vaccination results in long-lasting cellular immunity and increased immunogenicity [Hickling and Jones, 2009]. Human skin (epidermis and dermis) is rich in antigen-presenting cells (APCs), including Langerhans cells and dermal dendritic cells (DCs). Intradermal vaccination is known to be 5- to 10-times more effective than subcutaneous or intramuscular vaccination because it targets the APCs [Hickling and Jones, 2009], and such targeting also activates the T cell immunity pathway for long-lasting immunity. By intradermal injection, c-srRNA is predominantly taken up by skin APCs, wherein it replicates, produces antigen, digests the antigen into peptides, and presents these peptides to T cells (FIG. 1). The peptides presented through this pathway stimulates MHC-I-restricted CD8+ killer T cells. In an alternative pathway, APCs also take antigens produced by nearby skin cells The peptides presented through this pathway stimulate MHC-II-restricted CD4+ Helper T cells.

### Issues for Intradermal Injection and Our Solutions

Here are potential issues that we have identified and the solutions that our c-srRNA platform offers.
(1) A key unrecognized hurdle for the application of srRNA as an intradermal vaccine platform is that both mRNA and srRNA do not express antigen well at skin temperature [PCT/US20/67506]. Unintuitively, the temperature of the human skin is lower (about 30-35° C.) than human core body temperature (about 37° C.); this means that vectors and platforms developed at 37° C. are not optimal for intradermal injection. One innovation of our c-srRNA platform is that it expresses antigen strongly at skin temperature [PCT/US20/67506]. Furthermore, this temperature-control also minimizes the safety risk caused by unintended systemic distribution of c-srRNA because c-srRNA becomes inactivated once its temperature increases above its permissive threshold (when it moves closer to the core of the body). In other words, the c-srRNA platform expresses antigen the best for intradermal injection compared to mRNA and srRNA, and it additionally has safety features: the vector's ability to spread and become produced in other areas of a subject's body is limited or inactivated.
(2) Another challenge for intradermal vaccination is the lack of suitable additives. Because adjuvants such as aluminum-salt and oil-in-water are too reactogenic locally when delivered by the intradermal route, no adjuvant has been incorporated into clinically approved intradermal vaccines, resulting in lower immunogenicity [Hickling and Jones, 2009]. Lipid Nanoparticles (LNPs) used for mRNA and srRNA vaccines, which are administered intramuscularly, are also oil-in-water, which may cause skin reactogenicity and increase risk of allergic reactions to LNP components such as PEG. Our c-srRNA platform is a solution to this problem since it is injected as naked c-srRNA (no LNPs, no adjuvants). First, self-replication of RNAs inside cells, especially APCs, induces the strong innate immunity, which substitutes the major functions of adjuvants. Second, data in the literature and of our own demonstrates that, specifically for intradermal injection, naked mRNA/srRNA is equally efficient to produce an antigen compared to electroporation of mRNA/srRNA [Johansson et al, 2012] and mRNA/srRNAs combined with LNPs [Golombek et al., 2018].
(3) A third challenge is the limited number of precedents for intradermal vaccines. Only the BCG vaccine has been administered intradermally on a routine basis. One way we lower the hurdle for adopting intradermal injection is by using specialized devices such as the MicronJet600 (NanoPass) and Immucise (Terumo), which are now available to enable easy, consistent intradermal injection. These devices are also good candidates for large-scale production and deployment. However, due to a relatively high cost of these special devices, an intradermal injection by the Mantoux technique using a standard needle and syringe is also an option.

### Design of Suitable Antigens

A tumor-associated antigens (TAA) is expressed in tumor cells, but also expressed in embryonic cells or expressed at a low level in normal cells. The National Cancer Institute selected 75 cancer antigens that are suitable for a target of cancer therapy (Cheever et al., 2009). For example, Wilms tumor 1 (WT1) ranked as the most promising among the 75 cancer antigens identified by the National Cancer Institute (Cheever et al., 2009). WT1 is expressed in a broad range of tumors, but expressed only in embryonic tissues and very limited cell types in adults. For examples, WT1 is expressed in most leukemia (AML, ALL), pancreatic cancer, lung carcinomas, and Glioblastoma. WT1 peptides have been used as an antigen for cancer vaccines in many preclinical and clinical trials. The use of WT1 is shown in EXAMPLE 1. The list also contains NY-ESO-1 (EXAMPLE 2) and MAGEA3 (EXAMPLE 3). Any TAA can be used as an antigen for cancer vaccines based on our c-srRNA platform. It is also possible to use any combination of these TAAs as a fusion protein or proteins expressed separately (EXAMPLE 4).

Recently, it has become common to perform genome sequencing of tumor cells derived from patients. Such efforts often identify protein products or peptides that are unique to tumors due to the mutations in their genomes. These Tumor-specific antigens (TSA), also called neoantigens, are ideal targets for cancer vaccine. A single TSA or a fusion of more than one TSA can be used as an antigen for cancer vaccines based on our c-srRNA platform (EXAMPLE 5).

### Chitosan-Enhancement of Gene Expression In Vivo

An RNase inhibitor (a protein purified from human placenta) slightly enhances the immunogenicity against an antigen encoded on c-srRNA, most likely by enhancing expression of the antigen from the c-srRNA in vivo when intradermally injected into mice (see e.g., FIG. 25C of WO 2021/138447 A1). The RNase inhibitor may protect c-srRNA from RNase-mediated degradation in vivo. However, it is desirable to find an alternative agent that can enhance expression of a gene of interest (GOI) in vivo for therapeutics purposes, as it is difficult to use a protein-based RNase inhibitor as an excipient in injectable products.

A low molecular weight chitosan (molecular weight ˜6 kDa) was shown to inhibit the activity of RNase with the inhibition constants in the range of 30-220 nM (Yakovlev et al., Biochem Biophys Res Commun, 357 (3): 584-8, 2007). Two different chitosan oligomers were recently tested: chitosan oligomer (CAS No. 9012-76-4; molecular weight ≤5 kDa, ≥75% deacetylated: Heppe Medical Chitosan GmbH: Product No. 44009), and chitosan oligosaccharide lactate (CAS No. 148411-57-8; molecular weight about 5 kDa, >90% deacetylated: Sigma-Aldrich: Product No. 523682). Surprisingly, even a very low level of chitosan oligomers, as low as 0.001 μg/mL (about 0.2 nM: about 1/100 of the inhibition constant discovered by Yakovlev et al., supra, 2007) was found to be able to enhance the expression of luciferase encoded on c-srRNA by ˜10-fold (data not shown). Similar enhancement of the GOI expression was achieved by chitosan oligomers for up to 0.5 μg/mL and by chitosan oligosaccharide lactate at 0.1 μg/mL.

Chitosan has been used as a nucleotide (DNA and RNA) delivery vector, as it can form complexes or nanoparticles (reviewed in Buschmann et al., Adv Drug Deliv Rev, 65 (9): 1234-70, 2013; and Cao et al., Drugs, 17:381, 2019). However, it is worth noting that the enhancement of the GOI expression by chitosan oligomers is unlikely to be mediated by the nanoparticle or the complex formation of c-srRNA and chitosan oligomers. First, such a low concentration of chitosan oligomers does not allow the complex formation with RNA. Second, chitosan oligomers are added to c-srRNA immediately before the intradermal injection, and thus, there is not sufficient time to form the complex.

As the chitosan oligomers enhance expression of the GOI in vivo at much lower concentrations compared to the effective concentration as an RNase inhibitor in vitro (Yakovlev et al., supra, 2007), it is conceivable that this enhanced GOI expression by chitosan oligomers may not be mediated by its RNase inhibition mechanism. For example, chitosan oligomers may facilitate the incorporation of c-srRNA into cells, and thereby may enhance the expression of GOI from c-srRNA. Nonetheless, this surprising discovery should provide an effective means to enhance the in vivo therapeutic expression of GOI encoded on c-srRNA.

### Enumerated Embodiments

1. A composition for stimulating an immune response against a cancer antigen in a mammalian subject, comprising an excipient, and a temperature-sensitive self-replicating RNA comprising an open reading frame (ORF) encoding a fusion protein, and an Alphavirus replicon lacking a viral structural protein coding region, wherein the ORF comprises from 5′ to 3′:
   (i) a nucleotide sequence encoding a mammalian signal peptide; and
   (ii) a nucleotide sequence encoding a cancer antigen,
   wherein the temperature-sensitive self-replicating RNA is capable of expressing the fusion protein at a permissive temperature but not at a non-permissive temperature.
2. The composition of embodiment 1, wherein the cancer antigen comprises a tumor-associated antigen (TAA).
3. The comprises of embodiment 2, wherein the TAA comprises a WT1 antigen, a NY-ESO-1 antigen, a MAGEA3 antigen, a BIRC5 (SURVIVIN) antigen, a PRAME antigen, or a combination thereof.
4. The composition of embodiment 2, wherein the TAA comprises a WT1 antigen.
5. The composition of embodiment 4, wherein the amino acid sequence of the WT1 antigen comprises SEQ ID NO:2, or the amino acid sequence at least 95%, 96%, 97%, 98% or 99% identical to SEQ ID NO:2.
6. The composition of embodiment 2, wherein the TAA is a TAA fusion protein comprising a WT1 antigen, a NY-ESO-1 antigen, a MAGEA3 antigen, a BIRC5 antigen, and a PRAME antigen.
7. The composition of embodiment 6, wherein the amino acid sequence of the TAA fusion protein comprises SEQ ID NO:7, or the amino acid sequence at least 95%, 96%, 97%, 98% or 99% identical to SEQ ID NO:7.
8. The composition of embodiment 2, wherein the TAA comprises a BIRC5 antigen.
9. The composition of embodiment 8, wherein the amino acid sequence of the BIRC5 antigen comprises SEQ ID NO:3, or the amino acid sequence at least 95%, 96%, 97%, 98% or 99% identical to SEQ ID NO:3.
10. The composition of embodiment 2, wherein the TAA comprises a NY-ESO-1 antigen.
11. The composition of embodiment 10, wherein the amino acid sequence of the NY-ESO-1 antigen comprises SEQ ID NO:4, or the amino acid sequence at least 95%, 96%, 97%, 98% or 99% identical to SEQ ID NO:4.
12. The composition of embodiment 2, wherein the TAA comprises a MAGEA3 antigen.
13. The composition of embodiment 12, wherein the amino acid sequence of the MAGEA3 antigen comprises SEQ ID NO:5, or the amino acid sequence at least 95%, 96%, 97%, 98% or 99% identical to SEQ ID NO:5.
14. The composition of embodiment 2, wherein the TAA comprises a PRAME antigen.
15. The composition of embodiment 14, wherein the amino acid sequence of the PRAME antigen comprises SEQ ID NO:6, or the amino acid sequence at least 95%, 96%, 97%, 98% or 99% identical to SEQ ID NO:6.
16. The composition of embodiment 1, wherein the cancer antigen comprises a neoantigen.
17. The composition of any one of embodiments 1-16, wherein the mammalian signal peptide is a signal peptide of a surface protein expressed in mammalian antigen presenting cells.
18. The composition of embodiment 17, wherein the mammalian signal peptide is a CD5 signal peptide and the amino acid sequence of the CD5 signal peptide comprises SEQ ID NO:1, or the amino acid sequence at least 90% or 95% identical to SEQ ID NO: 1.
19. The composition of any one of embodiments 1-18, wherein the Alphavirus is selected from the group consisting of a Venezuelan equine encephalitis virus, a Sindbis virus, and a Semliki Forrest virus.
20. The composition of embodiment 19, wherein the Alphavirus is a Venezuelan equine encephalitis virus.
21. The composition of any one of embodiments 1-20, wherein the Alphavirus replicon comprises a nonstructural protein coding region with an insertion of 12-18 nucleotides resulting in expression of a nonstructural Protein 2 (nsP2) comprising from 4 to 6 additional amino acids between beta sheet 5 and beta sheet 6 of the nsP2.
22. The composition of embodiment 21, wherein the additional amino acids comprise the sequence of SEQ ID NO: 14 (TGAAA).
23. The composition of embodiment 22, wherein the amino acid sequence of the nsP2 comprises SEQ ID NO:12.
24. The composition of embodiment 23, wherein the amino acid sequence of the nsP2 comprises one sequence selected from the group consisting of SEQ ID NO:9, SEQ ID NO:10, and SEQ ID NO:11.
25. The composition of embodiment 24, wherein the amino acid sequence of the nsP2 comprises SEQ ID NO:11.
26. The composition of any one of embodiment 1-25, wherein the permissive temperature is from 30° C. to 36° C., or 31° C. to 35° C., or 32° C. to 34° C., or 33° C.±0.5° C., and the non-permissive temperature is 37° C.±0.5° C., optionally wherein the permissive temperature is from 31° C. to 35° C. and the non-permissive temperature is at least 37° C.±0.5° C.
27. The composition of any one of embodiment 1-26, wherein the composition does not comprise lipid nanoparticles.
28. The composition of any one of embodiments 1-27, wherein the composition further comprises chitosan.
29. A method for stimulating an immune response against a cancer antigen in a mammalian subject, comprising administering the composition of any one of embodiments 1-28 to a mammalian subject so as to stimulate an immune response against the cancer antigen in the mammalian subject.
30. The method of embodiment 29, wherein the composition is administered intradermally.
31. The method of embodiment 29 or embodiment 30, wherein the immune response comprises a cellular immune response reactive with mammalian cells expressing the cancer antigen.
32. The method of embodiment 31, wherein the cellular immune response comprises one or both of a cancer antigen-specific cytotoxic T lymphocyte response and a cancer antigen-specific helper T lymphocyte response.
33. The method of embodiment 32, wherein the immune response further comprises a humoral immune response reactive with the cancer antigen.
34. The method of any one of embodiments 29-33, wherein the mammalian subject is a human subject.
35. A kit comprising:
   (i) the composition of any one of embodiments 1-28; and
   (ii) a device for intradermal delivery of the composition to a mammalian subject.
36. The kit of embodiment 35, wherein the device comprises a syringe and a needle.

### EXAMPLES

Abbreviations: APC (antigen presenting cell); BIRC5 (baculoviral IAP repeat containing 5 or SUR VIVIN); IL-4 (interleukin-4); IFN-γ (interferon gamma); MAGEA3 (melanoma-associated antigen 3); ORF (open reading frame); PBO (placebo); NY-ESO-1 (New York esophageal squamous cell carcinoma 1 or CTAGIB); PRAME (preferentially expressed antigen in melanoma); SFC (spot-forming cells); srRNAts (temperature-sensitive, self-replicating RNA or c-srRNA temperature-controllable, self-replicating RNA); TAA (tumor-associated antigen); TSA (tumor-specific antigen); and WT1 (Wilms tumor 1).

### Example 1. Immunotherapy Against Tumors Expressing WT1

This example describes the finding that the human Wilms tumor 1 (WT1) protein induces a potent cellular immune response in BALB/c mice when expressed from an intradermally-injected, temperature-controllable, self-replicating RNA. Strikingly, the EXG-5101 RNA construct induces elimination of mouse mammary tumor cells expressing human WT1 in a syngeneic mouse cancer model.

### Materials and Methods

### BALB/c Inbred Female Mice.

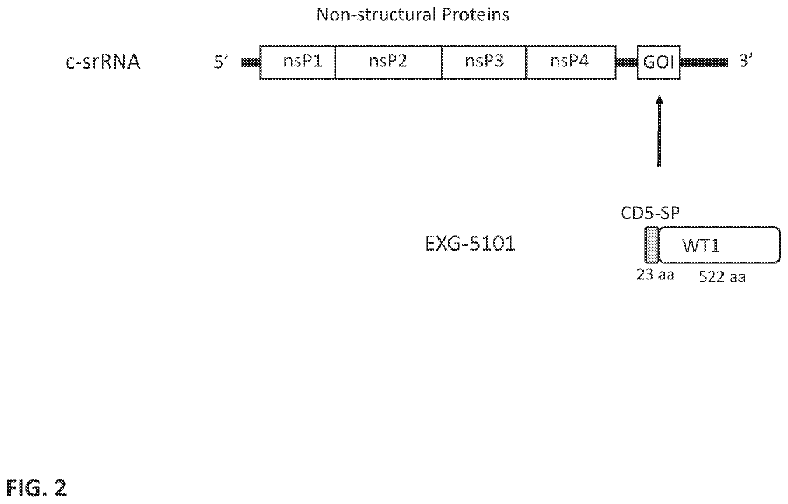

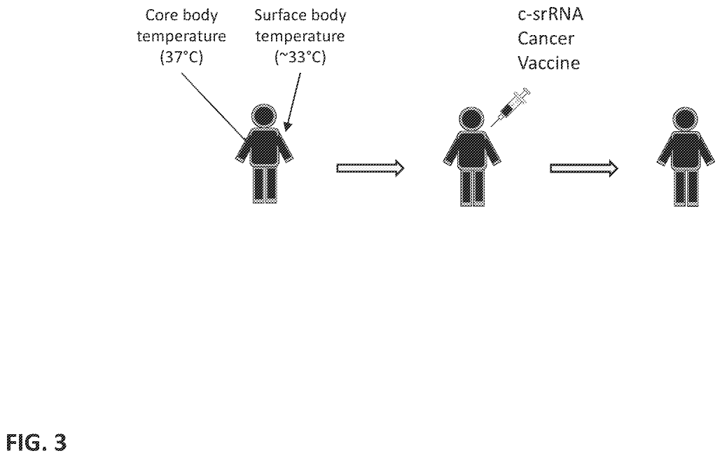

EXG-5101 mRNA was produced by in vitro transcription of a temperature-controllable self-replicating RNA vector (srRNA1ts2 [PCT/US2020/067506]) encoding a fusion protein comprising the human CD5 signal peptide fused to the human WT1 protein (FIG. 2). The WT1 protein of EXG-5101 is encoded by Isoform D, which starts with a non-AUG (CUG) translation initiation codon.

4T1 mammary tumor cells (ATCC No. CRL-2539) were derived from a BALB/c mouse and are known to mimic human breast cancer (Stage IV)

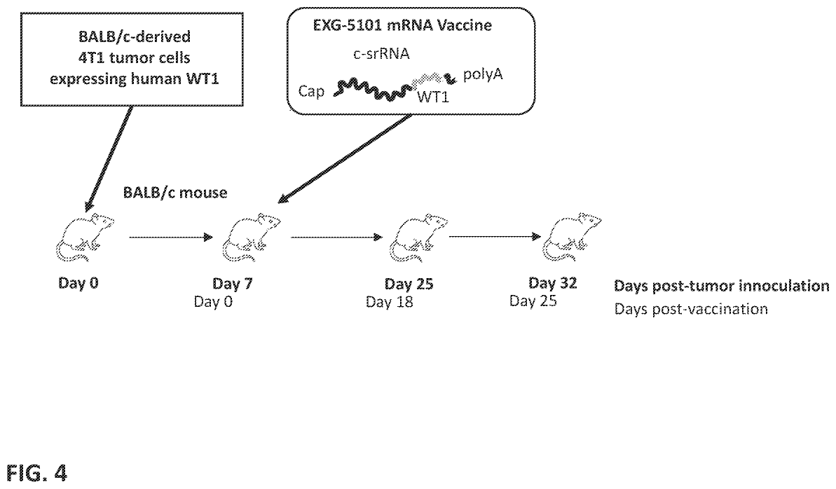

FIG. 4 illustrates the experimental procedure. 4T1 tumor cells were transfected with a plasmid DNA encoding a human Wilms tumor 1 (WT1) protein isoform D (NM_024426.6) driven by a CMV promoter, as well as a neomycin-resistance gene as a selectable marker. Stable transformants of 4TI cells expressing human WT1 were isolated by G418 selection. The cells were injected into a mammary fat pad of a BALB/c mouse (Day 0 post-tumor inoculation). On Day 7, either placebo (PBO), 5 μg, or 25 μg of EXG-5101 mRNA was intradermally administered (Day 0 post-vaccination). Tumor size was measured on Day 5, Day 8 (Day 0 post-vaccination), Day 25 (Day 18 post-vaccination), and Day 32 (Day 25 post-vaccination).

### Results and Conclusion

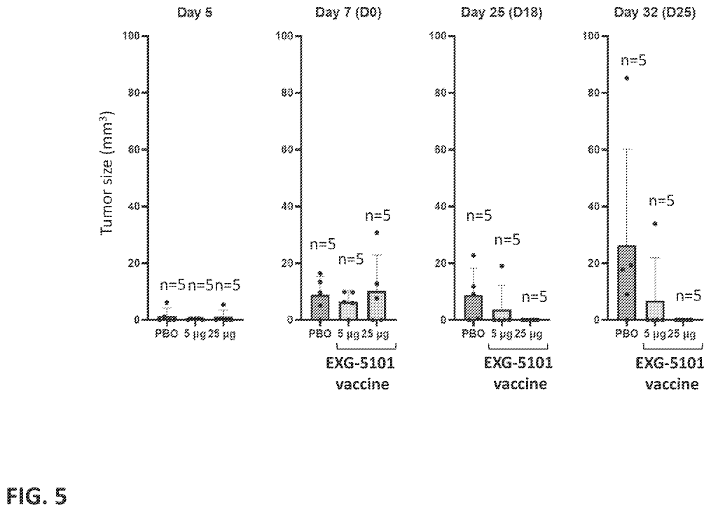

FIG. 5 shows the growth of tumors in BALB/c mice injected with a placebo (PBO), 5 μg, or 25 μg of EXG-5101 mRNA vaccine. The average and standard deviation (error bars) of five mice (n=5) are shown for each group. By Day 7 post-tumor inoculation, all three groups of mice developed tumors. However, by Day 25 (Day 18 post-vaccination), the tumor growth was suppressed in mice injected with EXG-5101 mRNA in a dose-dependent manner, whereas tumors continued to grow in mice injected with the placebo.

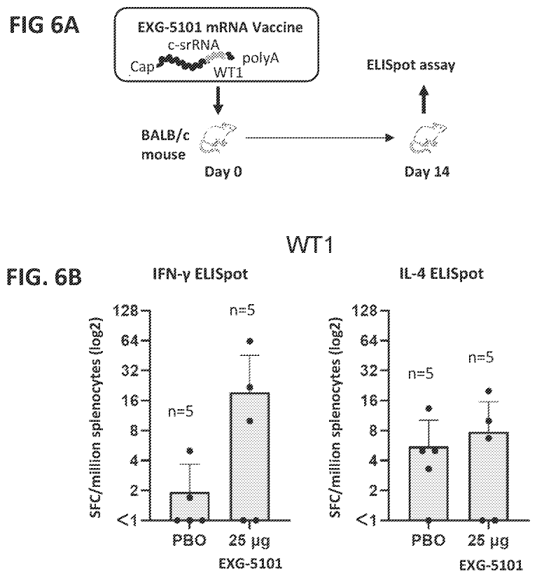

FIG. 6A-B shows induction of a tumor-associated antigen-reactive cellular immune response by intradermal injection of the EXG-5101 mRNA. As shown in FIG. 6A, BALB/c mice were intradermally injected with either 25 μg of EXG-5101 or placebo (PBO) on day 0. Splenocytes were collected from these mice on day 14 and used for ELISpot assays. FIG. 6B shows the results of ELISpot assays as the frequency of IFN-γ or IL-4 spot-forming cells (SFC) per 1×10\hat{ }6 splenocytes that were stimulated by a pool of 110 peptides that covers the human WT1 protein (15 mers with 11 amino acid overlaps). IFN-γ-secreting cells represent CD8+ T cells and CD4+ Th1 cells, which are regarded as cell-mediated (cellular) immune responses, whereas IL-4-secreting cells represent CD4+ Th2 cells. Accordingly, the results indicate that EXG-5101 induced cellular immunity against human WT1 protein.

In conclusion, the intradermally-injected EXG-5101 mRNA immunotherapeutic suppresses tumor growth or reduces tumor size of WT1-expressing tumors in a dose-dependent manner in a syngeneic mouse model of breast cancer. In addition, the intradermally-injected EXG-5101 mRNA immunotherapeutic induces cellular immunity against human WT1 protein in a mouse model.

### Example 2. Immunotherapy Against Tumors Expressing NY-ESO-1

This example describes assessing whether intradermally-injected c-srRNA encoding human NY-ESO-1 is able to induce a cellular immune response against mouse mammary tumor cells expressing human NY-ESO-1 in syngeneic mouse cancer model.

### Materials and Methods

### BALB/c Inbred Female Mice.

c-srRNA-NY-EOS1 mRNA is produced by in vitro transcription of a temperature-controllable, self-replicating RNA vector (srRNA1ts2 [PCT/US20/67506]) encoding a fusion protein comprising the human CD5 signal peptide fused to the human NY-ESO-1 protein. NY-ESO-1 is also known as Cancer/testis antigen 1B (CTAGIB) (NM_001327).

4T1 mammary tumor cells (ATCC No. CRL-2539) were derived from a BALB/c mouse and are known to mimic human breast cancer (Stage IV).

4T1 tumor cells are transfected with a plasmid DNA encoding a human NY-ESO-1, also known as Cancer/testis antigen 1B (CTAGIB) (NM_001327) driven by a CMV promoter, as well as a neomycin-resistance gene as a selectable marker. Stable transformants of 4TI cells expressing human NY-ESO-1 gene are isolated by G418 selection. The cells are injected into a mammary fat pad of a BALB/c mouse (Day 0 post-tumor inoculation). On Day 7, either placebo (PBO), 5 μg, or 25 μg of c-srRNA-NY-ESO-1 mRNA is intradermally administered (Day 0 post-vaccination). Tumor size is measured at several time points post vaccination.

### Results and Conclusion

Intradermally-injected c-srRNA-NY-ESO-1 mRNA immunotherapeutic is contemplated to suppress tumor growth or reduce tumor size of NY-ESO-1-expressing tumors in a dose-dependent manner in a syngeneic mouse model of breast cancer.

### Example 3. Immunotherapy Against Tumors Expressing MAGEA3

This example describes assessing whether intradermally-injected c-srRNA encoding human MAGE family member A3 (MAGEA3) is able to induce a cellular immune response against mouse mammary tumor cells expressing human MAGEA3 in syngeneic mouse cancer model.

### Materials and Methods

### BALB/c Inbred Female Mice.

c-srRNA mRNA-MAGEA3 is produced by in vitro transcription of a temperature-controllable, self-replicating RNA vector (srRNA1ts2 [PCT/US20/67506]) encoding a fusion protein comprising the human CD5 signal peptide fused to the human MAGE family member A3 (MAGEA3) protein (NM_005362).

4T1 mammary tumor cells (ATCC No. CRL-2539) were derived from a BALB/c mouse and are known to mimic human breast cancer (Stage IV).

4T1 tumor cells are transfected with a plasmid DNA encoding a human MAGEA3 (NM_005362) driven by a CMV promoter, as well as a neomycin-resistance gene as a selectable marker. Stable transformants of 4T1 cells expressing human MAGEA3 are isolated by G418 selection. The cells are injected into a mammary fat pad of a BALB/c mouse (Day 0 post-tumor inoculation). On Day 7, either placebo (PBO), 5 μg, or 25 μg of c-srRNA-MAGEA3 mRNA is intradermally administered (Day 0 post-vaccination). Tumor size is measured at several time points post vaccination.

### Results and Conclusion

Intradermally-injected c-srRNA-MAGEA3 mRNA immunotherapeutic is contemplated to suppress tumor growth or reduce tumor size of MAGEA3-expressing tumors in a dose-dependent manner in a syngeneic mouse model of breast cancer.

### Example 4. Immunotherapy Against Tumors Expressing Two or More Tumor-Associated Antigens (TAAs)

This example describes the finding that intradermally-injected c-srRNA encoding a fusion protein comprising WT1, NY-ESO-1, BIRC5, MAGEA3, and PRAME induces a potent cellular immune response in BALB/c mice against TAAs of the fusion protein.

### Materials and Methods

### BALB/c Inbred Female Mice.

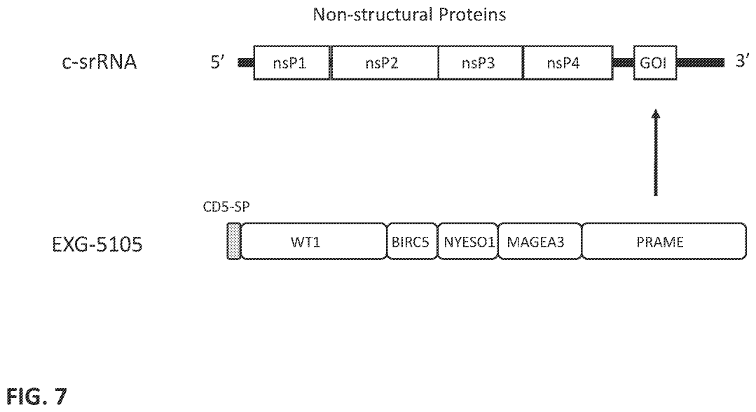

FIG. 7 shows a schematic diagram of EXG-5105 vaccine, which is a c-srRNA mRNA (srRNA1ts2 [PCT/US20/67506]) encoding a fusion protein of human WT1, NY-ESO-1, BIRC5, MAGEA3, and PRAME with a signal peptide sequence derived from human CD5 gene.

4T1 mammary tumor cells derived from BALB/c (ATCC: CRL-2539), which is known to mimic human breast cancer (Stage IV).

4T1 tumor cell line was transfected with three plasmid DNAs, encoding a human WT1, BIRC5, NY-ESO-1, MAGEA3, and PRAME, respectively, driven by a CMV promoter and a selectable marker against G418 (neomycin). The stable transformant of 4T1 cells expressing human WT1, BIRC5, NY-ESO-1, MAGEA3, and PRAME, was isolated after G418 selection. The cells were injected into a mammary fat pad of a BALB/c mouse. Either placebo (PBO), 5 μg, or 25 μg of EXG-5105 mRNA vaccine was intradermally administered. Subsequently, tumor sizes were measured.

### Results and Conclusion

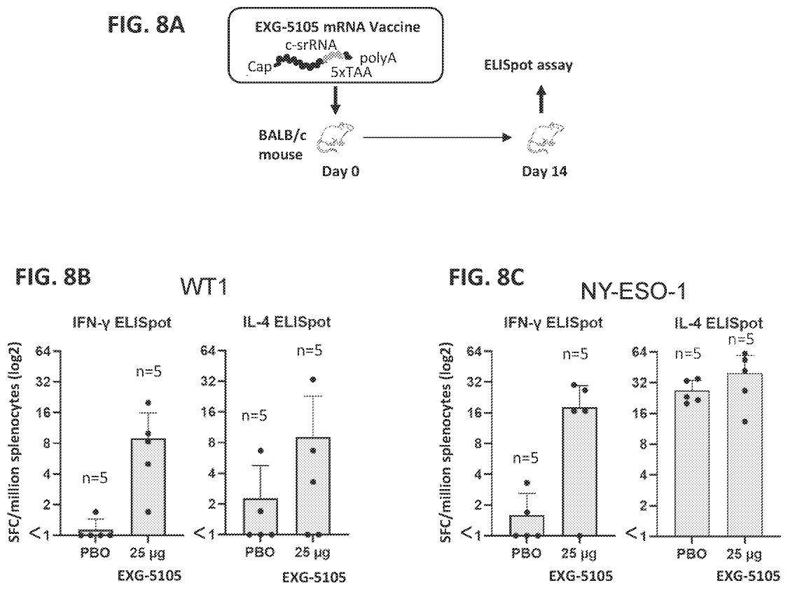

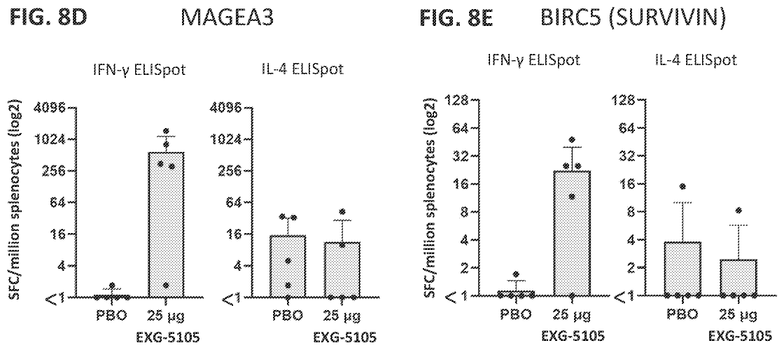

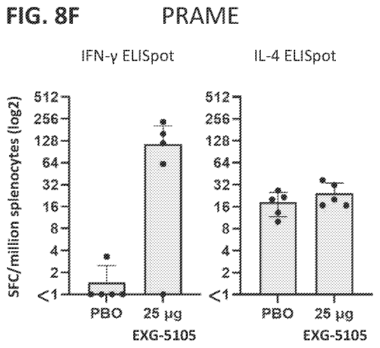

FIG. 8A shows the experimental procedure to examine the immunogenicity of EXG-5105 mRNA vaccine. BALB/c mice received the intradermal injection of either 25 μg of EXG-5105 or placebo (PBO) on day 0. Splenocytes were collected from these mice on day 14 and used for ELISpot assays. EXG-5105 encodes a fusion protein comprising human WT1, NY-ESO-1, BIRC5, MAGEA3, and PRAME. As such, the intradermal injection of EXG-5105 is expected to induce cellular immunity against all five of these TAAs at the same time. Indeed, the results shown in FIG. 8B-8F indicate that this was the case. FIG. 8B shows the results of ELISpot assays as the frequency of IFN-γ or IL-4 spot-forming cells (SFC) per 1×10\hat{ }6 splenocytes that were stimulated by a pool of 110 peptides that covers the human WT1 protein (15 mers with 11 amino acid overlaps). IFN-γ-secreting cells represent CD8+ T cells and CD4+ Th1 cells, which are indicative of cell-mediated (cellular) immune responses, whereas IL-4-secreting cells represent CD4+ Th2 cells. Accordingly, the results indicate that EXG-5105 induced cellular immunity against a human WT1 protein. Similarly, FIG. 8C shows the results of ELISpot assays as the frequency of IFN-γ or IL-4 spot-forming cells (SFC) per 1×10\hat{ }6 splenocytes that were stimulated by a pool of peptides that covers the human NY-ESO-1 protein (15 mers with 11 amino acid overlaps). The results indicate that EXG-5105 induced cellular immunity against the human NY-ESO-1 protein, as well as the human WT1 protein. Similarly, FIG. 8D, FIG. 8E, and FIG. 8F show the frequency of cytokine (left, interferon-gamma [IFN-γ]; right, Interleukin-4 [IL-4)]) spot-forming cells (SFC) per 1×10\hat{ }6 splenocytes that were stimulated by a pool of peptides that covers the human MAGEA3 protein, human BIRC5 (SURVIVIN) protein, and human PRAME protein, respectively. Interestingly, more potent cellular immune responses were elicited against MAGEA3 and PRAME, both of which are expressed rather exclusively in tumors and testes, than against WT1, NY-ESO-1, and BIRC5, all of which are expressed in tumors, as well as in some other tissues.

In conclusion, the intradermally-injected EXG-5105 mRNA immunotherapeutic induces cellular immunity against distinct components of a fusion protein in a syngeneic mouse cancer model. Additionally, the intradermally-injected EXG-5105 mRNA vaccine is expected to suppress growth of tumor cells expressing human WT1, NY-ESO-1, BIRC5, MAGEA3, and PRAME in vivo.

### Example 5. Immunotherapeutic Against Tumors Expressing a Tumor-Specific Antigen (TSA)

This example describes the finding that intradermally-injected srRNAts encoding for a neoantigen induces a cellular immune response in BALB/c mice against the neoantigen in syngeneic mouse cancer model.

### Materials and Methods

### BALB/c Inbred Female Mice.

srRNAts mRNA (srRNA1ts2 [PCT/US20/67506]) encoding for a neoantigen with a signal peptide sequence derived from human CD5 gene.

4T1 mammary tumor cells derived from BALB/c (ATCC: CRL-2539), which is known to mimic human breast cancer (Stage IV).

4T1 tumor cell line was transfected with three plasmid DNAs, encoding a human neoantigen driven by a CMV promoter and a selectable marker against G418 (neomycin). The stable transformant of 4Tl cells expressing human neoantigen was isolated after G418 selection. The cells were injected into a mammary fat pad of BALB/c mouse. Either placebo (PBO), 5 μg, or 25 μg of srRNAts-neoantigen mRNA vaccine was intradermally administered. Subsequently, tumor sizes were measured.

### Results and Conclusion

Intradermally-injected srRNAts-neoantigen mRNA vaccine suppresses the growth of tumor cells expressing human neoantigen and eliminates the tumors in a dose-dependent manner in syngeneic mouse cancer model.

### Example 6. Comparison of Self-Replicating RNAs for T-Cell Inducibility

This example describes the finding that intradermally-injected srRNAts constructs encoding an antigen induce a cellular immune response in mice against the antigen.

### Materials and Methods

C57BL/6 mice.

Three different temperature-controllable self-replicating RNA vectors (c-srRNA) and a control self-replicating RNA vector (c-srRNA) were tested. Characteristics of the srRNAs are summarized in Table 6-1. IFN-α/β sensitivity of the parental VEEV strains was previously reported (Spotts et al., J Viol, 72:10286-10291, 1998). c-srRNAl was based on the TRD strain of VEEV but modified to have a A16D substitution (TC83 mutation) and a P778S substitution. c-srRNA3 was also based on the TRD strain of VEEV but without the A16D and P778S substitutions. srRNA4 was based on the V198 strain of VEEV, which was isolated from a human. All three c-srRNA vectors include the same 5 amino acid insertion within the nsP2 protein of VEEV for temperature-controllability, as previously described (see U.S. Pat. No. 11,421,248 to Ko, Examples 3, 21 and 22 incorporated herein by reference). All four srRNAs encode an antigen (SARS-COV-2 spike protein receptor binding domain) lacking a signal peptide sequence.

TABLE 6-1
srRNA Characteristics

| RNA | ts-mutant | VEEV |
|---|---|---|
| srRNA0 | no | TRD |
| c-srRNA1 | yes | TRD/TC-83 |
| c-srRNA3 | yes | TRD |
| c-srRNA4 | yes | V198 |

The nucleotide sequences of the VEEV genomes are disclosed in GenBank: TRD strain as GenBank No. L01442.2; and TC-83 strain as GenBank No. L01443.1. The amino acid sequences of the nsP2 proteins of the srRNAs are disclosed herein: srRNA0 (SEQ ID NO: 13); c-srRNAI (SEQ ID NO:9); c-srRNA3 (SEQ ID NO:10); c-srRNA4 (SEQ ID NO:11); and c-srRNA consensus (SEQ ID NO:12).

Preparation of srRNA. All srRNAs were produced by in vitro transcription. NEB 10-beta competent E. coli (C3019H/C30191) was transformed with a plasmid DNA and cultured in Luria Broth containing 100 μg/mL ampicillin. Purified plasmid DNA was linearized by MluI. In vitro transcription (IVT) of c-srRNA with Cap1 and poly A was performed using in vitro transcription of a plasmid DNA using T7 RNA polymerase with Cleancap AU (Trilink) according to the manufacturer's protocol.

Injection of srRNA into mouse skin. Mice were randomly divided into groups, and the fur on the hindlimb was shaved to expose the skin one-day prior injection. 5 μg or 25 μg of srRNA reconstituted in Lactated ringer's (LR) solution was intradermally injected onto the shaved skin.

### Results and Conclusion

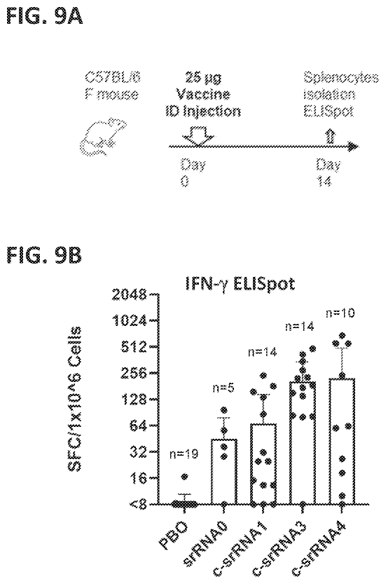

C57BL/6 mice received one of the srRNAs as naked RNA (without lipid nanoparticles or transfection reagents) or a placebo by intradermal injection (FIG. 9A). As expected, the cellular immunity assessed by the presence of antigen-specific IFN-γ-secreting T cells was already induced by day 14 post-vaccination (FIG. 9B). The T-cell response induced by C-srRNA1 was stronger than the response induced by the standard, non-temperature-controllable srRNAO. In addition, the T-cell responses induced by both c-srRNA3 and c-srRNA4 were stronger than the responses induced by srRNAO and c-srRNA1. Strikingly, the T-cell responses induced by both c-srRNA3 and c-srRNA4 were about 3-fold higher than the responses induced by c-srRNA1. This difference is contemplated to be due to the parental VEEV sequences of c-srRNA3 and c-srRNA4 being more resistant to suppression by type I interferons than the parental VEEV sequence of c-srRNA1.

### REFERENCES

References pertaining to the present disclosure include: PCT/US2020/067506 of Elixirgen Therapeutics, Inc.; Brito et al., Mol Ther. 22 (12): 2118-2129, 2014; Cheever et al., Clin Cancer Res. 15:5323-5337, 2009; Golombek et al., Mol Ther Nucleic Acids. 11:382-392, 2018; Hickling et al., Intradermal Delivery of Vaccines: A review of the literature and the potential for development for use in low- and middle-income countries. PATH/WHO Aug. 27, 2009; Johanning et al., Nucleic Acids Res. 23 (9): 1495-501, 1995; and Johansson et al., PLOS One. 7 (1): e29732, 2012.

SEQUENCES

SEQ ID NO: 1
>Human CD5 Signal Peptide
MPMGSLQPLATLYLLGMLVASCLG
SEQ ID NO: 2
>Human Wilms tumor protein (NM_024426.6)
MDFLLLQDPASTCVPEPASQHTLRSGPGCLQQPEQQGVRDPGGIWAKLGAAEASAERLQGRRSRGASGSE
PQQMGSDVRDLNALLPAVPSLGGGGGCALPVSGAAQWAPVLDFAPPGASAYGSLGGPAPPPAPPPPPPPP
PHSFIKQEPSWGGAEPHEEQCLSAFTVHFSGQFTGTAGACRYGPFGPPPPSQASSGQARMFPNAPYLPSC
LESQPAIRNQGYSTVTEDGTPSYGHTPSHHAAQFPNHSFKHEDPMGQQGSLGEQQYSVPPPVYGCHTPTD
SCTGSQALLLRTPYSSDNLYQMTSQLECMTWNQMNLGATLKGVAAGSSSSVKWTEGQSNHSTGYESDNHT
TPILCGAQYRIHTHGVERGIQDVRRVPGVAPTLVRSASETSEKRPEMCAYPGCNKRYFKLSHLQMHSRKH
TGEKPYQCDEKDCERRFSRSDQLKRHQRRHTGVKPFQCKTCQRKESRSDHLKTHIRTHTGKTSEKPFSCR
WPSCQKKFARSDELVRHHNMHQRNMTKLQLAL
SEQ ID NO: 3
>Human BIRC5 (AKA SURVIVIN) protein (NM_001168)
MGAPTIPPAWQPFLKDHRISTFKNWPFLEGCACTPERMAEAGFIHCPTENEPDLAQCEFCFKELEGWEPD
DDPIEEHKKHSSGCAFLSVKKQFEELTLGEFLKLDRERAKNKIAKETNNKKKEFEETAEKVRRAIEQLAA
MD
SEQ ID NO: 4
>Human NY-ESO-1 protein (NM_001327)
MQAEGRGIGGSTGDADGPGGPGIPDGPGGNAGGPGEAGATGGRGPRGAGAARASGPGGGAPRGPHGGAAS
GINGCCRCGARGPESRLLEFYLAMPFATPMEAELARRSLAQDAPPLPVPGVLLKEFTVSGNILTIRLTAA
DHRQLQLSISSCLQQLSLIMWITQCFLPVFLAQPPSGQRR
SEQ ID NO: 5
>Human MAGEA3 protein (NM_005362)
MPLEQRSQHCKPEEGLEARGEALGLVGAQAPATEEQEAASSSSTLVEVTLGEVPAAESPDPPQSPQGASS
LPTTMNYPLWSQSYEDSSNQEEEGPSTEPDLESEFQAALSRKVAELVHFLLLKYRAREPVTKAEMLGSVV
GNWQYFFPVIFSKASSSLQLVEGIELMEVDPIGHLYIFATCLGLSYDGLLGDNQIMPKAGLLIIVLAIIA
REGDCAPEEKIWEELSVLEVFEGREDSILGDPKKLLTQHFVQENYLEYRQVPGSDPACYEFLWGPRALVE
TSYVKVLHHMVKISGGPHISYPPLHEWVLREGEE
SEQ ID NO: 6
>Human PRAME protein (NM_001291715)
MERRRLWGSIQSRYISMSVWTSPRRLVELAGQSLLKDEALAIAALELLPRELFPPLEMAAFDGRHSQTLK
AMVQAWPFTCLPLGVLMKGQHLHLETFKAVLDGLDVLLAQEVRPRRWKLQVLDLRKNSHQDFWTVWSGNR
ASLYSFPEPEAAQPMTKKRKVDGLSTEAEQPFIPVEVLVDLFLKEGACDELFSYLIEKVKRKKNVLRLCC
KKLKIFAMPMQDIKMILKMVQLDSIEDLEVTCTWKLPTLAKFSPYLGQMINLRRLLLSHIHASSYISPEK
EEQYIAQFTSQFLSLQCLQALYVDSLFFLRGRLDQLLRHVMNPLETLSITNCRLSEGDVMHLSQSPSVSQ
LSVLSLSGVMLTDVSPEPLQALLERASATLQDLVEDECGITDDQLLALLPSLSHCSQLTTLSFYGNSISI
SALQSLLQHLIGLSNLTHVLYPVPLESYEDIHGTLHLERLAYLHARLRELLCELGRPSMVWLSANPCPHC
GDRTFYDPEPILCPCFMPN
SEQ ID NO: 7
>ARTIFICIAL PROTEIN: FUSION OF WT1, BIRC5, NY-ESO-1, MAGEA3, and PRAME
proteins
LDFLLLQDPASTCVPEPASQHTLRSGPGCLQQPEQQGVRDPGGIWAKLGAAEASAERLQGRRSRGASGSE
PQQMGSDVRDLNALLPAVPSLGGGGGCALPVSGAAQWAPVLDFAPPGASAYGSLGGPAPPPAPPPPPPPP
PHSFIKQEPSWGGAEPHEEQCLSAFTVHFSGQFTGTAGACRYGPFGPPPPSQASSGQARMFPNAPYLPSC
LESQPAIRNQGYSTVTFDGTPSYGHTPSHHAAQFPNHSFKHEDPMGQQGSLGEQQYSVPPPVYGCHTPTD
SCTGSQALLLRTPYSSDNLYQMTSQLECMTWNQMNIGATLKGVAAGSSSSVKWTEGQSNHSTGYESDNHT
TPILCGAQYRIHTHGVERGIQDVRRVPGVAPTLVRSASETSEKRPFMCAYPGCNKRYFKLSHLQMHSRKH
TGEKPYQCDEKDCERRESRSDQLKRHQRRHTGVKPFQCKTCQRKFSRSDHLKTHTRTHTGKTSEKPESCR
WPSCQKKFARSDELVRHHNMHQRNMTKLQLALMGAPTLPPAWQPFLKDHRISTFKNWPFLEGCACTPERM
AEAGFIHCPTENEPDLAQCFFCFKELEGWEPDDDPIEEHKKHSSGCAFLSVKKQFEELTLGEFLKLDRER
AKNKIAKEINNKKKEFEETAEKVRRAIEQLAAMDMQAEGRGTGGSTGDADGPGGPGIPDGPGGNAGGPGE
AGATGGRGPRGAGAARASGPGGGAPRGPHGGAASGINGCCRCGARGPESRLLEFYLAMPFATPMEAELAR
RSLAQDAPPLPVPGVLIKEFTVSGNILTIRLTAADHRQLQLSISSCLQQLSLIMWITQCFLPVFLAQPPS
GQRRMPLEQRSQHCKPEEGLEARGEALGLVGAQAPATEEQEAASSSSTLVEVTLGEVPAAESPDPPQSPQ
GASSIPTTMNYPLWSQSYEDSSNQEEEGPSTFPDLESEFQAALSRKVAELVHFLLLKYRAREPVTKAEML
GSVVGNWQYFFPVIFSKASSSLQLVEGIELMEVDPIGHLYIFATCLGLSYDGLIGDNQIMPKAGLLIIVL
AIIAREGDCAPEEKIWEELSVLEVFEGREDSILGDPKKLLTQHFVQENYLEYRQVPGSDPACYEFLWGPR
ALVETSYVKVLHHMVKISGGPHISYPPLHEWVLREGEEMERRRLWGSIQSRYISMSVWTSPRRIVELAGQ
SLIKDEALAIAALELLPRELFPPLFMAAFDGRHSQTLKAMVQAWPFTCLPLGVLMKGQHLHLETFKAVLD
GLDVLLAQEVRPRRWKLQVLDLRKNSHQDEWTVWSGNRASLYSEPEPEAAQPMTKKRKVDGLSTEAEQPF
IPVEVLVDLFLKEGACDELESYLIEKVKRKKNVLRLCCKKLKIFAMPMQDIKMILKMVQLDSIEDLEVTC
TWKLPTLAKFSPYLGQMINLRRLLLSHIHASSYISPEKEEQYIAQFTSQFLSLQCLQALYVDSLFFLRGR
LDQLLRHVMNPLETLSITNCRLSEGDVMHLSQSPSVSQLSVISLSGVMLTDVSPEPLQALLERASATLQD
LVEDECGITDDQLLALLPSLSHCSQLTTLSFYGNSISISALQSLLQHLIGLSNLTHVLYPVPLESYEDIH
GTLHLERLAYLHARLRELLCELGRPSMVWLSANPCPHCGDRTFYDPEPILCPCEMPN
SEQ ID NO: 8
>ARTIFICIAL PROTEIN: A FUSION OF Human CD5 (signal peptide only), WT1,
BIRC5, NY-ESO-1, MAGEA3, and PRAME proteins
MPMGSLQPLATLYLLGMLVASCLGLDFLLLQDPASTCVPEPASQHTLRSGPGCLQQPEQQGVRDPGGIWA
KLGAAEASAERLQGRRSRGASGSEPQQMGSDVRDLNALLPAVPSLGGGGGCALPVSGAAQWAPVLDFAPP
GASAYGSLGGPAPPPAPPPPPPPPPHSFIKQEPSWGGAEPHEEQCLSAFTVHFSGQFTGTAGACRYGPFG
PPPPSQASSGQARMEPNAPYLPSCLESQPAIRNQGYSTVTFDGTPSYGHTPSHHAAQFPNHSFKHEDPMG
QQGSLGEQQYSVPPPVYGCHTPTDSCTGSQALLLRTPYSSDNLYQMTSQLECMTWNQMNLGATLKGVAAG
SSSSVKWTEGQSNHSTGYESDNHTTPILCGAQYRIHTHGVERGIQDVRRVPGVAPTLVRSASETSEKRPE
MCAYPGCNKRYFKLSHLQMHSRKHTGEKPYQCDEKDCERRESRSDQLKRHQRRHTGVKPFQCKTCQRKES
RSDHLKTHTRTHTGKTSEKPFSCRWPSCQKKFARSDELVRHHNMHQRNMTKLQLALMGAPTLPPAWQPFL
KDHRISTFKNWPFLEGCACTPERMAEAGFIHCPTENEPDLAQCFFCFKELEGWEPDDDPIEEHKKHSSGC
AFLSVKKQFEELTLGEFLKLDRERAKNKIAKETNNKKKEFEETAEKVRRAIEQLAAMDMQAEGRGTGGST
GDADGPGGPGIPDGPGGNAGGPGEAGATGGRGPRGAGAARASGPGGGAPRGPHGGAASGLNGCCRCGARG
PESRLLEFYLAMPFATPMEAELARRSLAQDAPPLPVPGVLLKEFTVSGNILTIRLTAADHRQLQLSISSC
LQQLSLIMWITQCFLPVFLAQPPSGQRRMPLEQRSQHCKPEEGLEARGEALGLVGAQAPATEEQEAASSS
STLVEVTLGEVPAAESPDPPQSPQGASSLPTTMNYPLWSQSYEDSSNQEEEGPSTFPDLESEFQAALSRK
VAELVHFLLLKYRAREPVTKAEMLGSVVGNWQYFFPVIFSKASSSLQLVFGIELMEVDPIGHLYIFATCL
GLSYDGLIGDNQIMPKAGLLIIVLAIIAREGDCAPEEKIWEELSVLEVFEGREDSILGDPKKLLTQHFVQ
ENYLEYRQVPGSDPACYEFLWGPRALVETSYVKVLHHMVKISGGPHISYPPLHEWVLREGEEMERRRLWG
SIQSRYISMSVWTSPRRLVELAGQSLLKDEALAIAALELLPRELFPPLFMAAFDGRHSQTLKAMVQAWPF
TCLPLGVLMKGQHLHLETFKAVLDGLDVLLAQEVRPRRWKLQVLDLRKNSHQDFWTVWSGNRASLYSEPE
PEAAQPMTKKRKVDGLSTEAEQPFIPVEVLVDLFLKEGACDELFSYLIEKVKRKKNVLRLCCKKLKIFAM
PMQDIKMILKMVQLDSIEDLEVTCTWKLPTLAKFSPYLGQMINLRRLLLSHIHASSYISPEKEEQYIAQF
TSQFLSLQCLQALYVDSLFFLRGRLDQLLRHVMNPLETLSITNCRLSEGDVMHLSQSPSVSQLSVLSLSG
VMLTDVSPEPLQALLERASATLQDLVEDECGITDDQLLALLPSLSHCSQLTTLSFYGNSISISALQSLLQ
HLIGLSNITHVLYPVPLESYEDIHGTLHLERLAYLHARLRELLCELGRPSMVWLSANPCPHCGDRTFYDP
EPILCPCEMPN
SEQ ID NO: 9
>ARTIFICIAL PROTEIN: c-SERNA1 nsP2
GSVETPRGLIKVTSYDGEDKIGSYAVLSPQAVIKSEKLSCIHPLAEQVIVITHSGRKGRY
AVEPYHGKVVVPEGHAIPVQDFQALSESATIVYNEREFVNRYLHHIATHGGALNTDEEYY
KTVKPSEHDGEYLYDIDRKQCVKKELVTGLGLIGELVDPPFHEFAYESLRTRPAAPYQVP
TIGVYGVPGSGKSGIIKSAVIKKDLVVSAKKENCAEIIRDVKKMKGLDVNARTVDSVLLN
GCKHPVETLYIDEAFACHAGTLRALIAIIRPKKAVLCGDPKQCGFENMMCLKVHENHEIC
TQVFHKSISRRCTKSVISVVSTLFYDKKMRTINPKETKIVIDTTGSTKPKQDDLILTCER
GWVKQLQIDYKGNEIMTAAASQGLTRKGVYAVRYKVNENPLYAPTSEHVNVLLTRTEDRI
VWKTLAGDPWIKTLTAKYPGNETATIEEWQAEHDAIMRHILERPDPTDVFQNKANVCWAK
ALVPVLKTAGIDMTTEQWNTVDYFETDKAHSAEIVLNQLCVRFFGLDLDSGLFSAPTVPL
SIRNNHWDNSPSPNMYGLNKEVVRQLSRRYPQLPRAVATGRVYDMNTGAAATGTLRNYDP
RINLVPVNRRLPHALVLHHNEHPQSDFSSFVSKLKGRTVLVVGEKLSVPGKMVDWLSDRP
EATFRARIDIGIPGDVPKYDIIFVNVRTPYKYHHYQQCEDHAIKLSMLTKKACLHLNPGG
TCVSIGYGYADRASESIIGAIARQFKFSRVCKPKSSLEETEVLFVFIGYDRKARTHNSYK
ISSTLTNIYTGSRLHEAGC
SEQ ID NO: 10
>ARTIFICIAL PROTEIN: C-srRNA3 nsP2
GSVETPRGLIKVTSYAGEDKIGSYAVLSPQAVLKSEKLSCIHPLAEQVIVITHSGRKGRY
AVEPYHGKVVVPEGHAIPVQDFQALSESATIVYNEREFVNRYLHHIATHGGALNTDEEYY
KTVKPSEHDGEYLYDIDRKQCVKKELVTGLGLTGELVDPPFHEFAYESLRTRPAAPYQVP
TIGVYGVPGSGKSGIIKSAVTKKDLVVSAKKENCAEIIRDVKKMKGLDVNARTVDSVLLN
GCKHPVETLYIDEAFACHAGTLRALIAIIRPKKAVLCGDPKQCGFENMMCLKVHENHEIC
TQVFHKSISRRCTKSVTSVVSTLFYDKKMRTINPKETKIVIDTTGSTKPKQDDLILTCER
GWVKQLQIDYKGNEIMTAAASQGLTRKGVYAVRYKVNENPLYAPTSEHVNVLLTRTEDRI
VWKTLAGDPWIKTLTAKYPGNFTATIEEWQAEHDAIMRHILERPDPTDVFQNKANVCWAK
ALVPVLKTAGIDMTTEQWNTVDYFETDKAHSAEIVLNQLCVRFFGLDLDSGLFSAPTVPL
SIRNNHWDNSPSPNMYGLNKEVVRQLSRRYPQLPRAVATGRVYDMNTGAAATGTLRNYDP
RINLVPVNRRLPHALVLHHNEHPQSDESSEVSKLKGRTVLVVGEKLSVPGKMVDWLSDRP
EATFRARLDLGIPGDVPKYDIIFVNVRTPYKYHHYQQCEDHAIKLSMLTKKACLHLNPGG
TCVSIGYGYADRASESIIGAIARQFKFSRVCKPKSSLEETEVLEVEIGYDRKARTHNPYK
LSSTLTNIYTGSRLHEAGC
SEQ ID NO: 11
>ARTIFICIAL PROTEIN: c-srRNA4 nsP2
GSVETPRGLIKVTSYAGEDKIGSYAVLSPQAVLKSEKLSCIHPLAEQVIVITHSGRKGRY
AVEPYHGKVVVPEGHAIPVQDFQALSESATIVYNEREFVNRYLHHIATHGGALNTDEEYY
KTVKPSEHDGEYLYDIDRKQCVKKELVTGLGLTGELVDPPFHEFAYESLRTRPAAPYQVP
TIGVYGVPGSGKSGIIKSAVIKKDLVVSAKKENCAEIIRDVKKMKGLDVNARTVDSVLLN
GCKHPVETLYIDEAFACHAGTLRALIAIIRPKKAVLCGDPKQCGFFNMMCLKVHFNHEIC
TQVFHKSISRRCTKSVTSVVSTLFYDKRMRTTNPKETKIEIDTTGSTKPKQDDLILTCER
GWVKQLQIDYKGNEIMTAAASQGLTRKGVYAVRYKVNENPLYAPTSEHVNVLLTRTEDRI
VWKTLAGDPWIKTLTAKYPGNFTATIEEWQAEHDAIMRHILERPDPTDVFQNKANVCWAK
ALVPVLKTAGIDMTTEQWNTVDYFETDKAHSAEIVLNQLCVRFFGLDLDSGLFSAPTVPL
SIRNNHWDNSPSPNMYGLNKEVVRQLSRRYPQLPRAVATGRVYDMNTGAAATGTLRNYDP
RINLVPVNRRLPHALVLHHNEHPQSDFSSFVSKLKGRTVLVVGEKLSVPGKKVDWLSDQP
EATFRARLDIGIPGDVPKYDIVFINVRTPYKYHHYQQCEDHAIKLSMLTKKACLHLNPGG
TCVSIGYGYADRASESIIGAIARQFKFSRVCKPKSSHEETEVLEVFIGYDRKARTHNPYK
LSSTLTNIYTGSRLHEAGC
SEQ ID NO: 12
>ARTIFICIAL PROTEIN: c-SERNA nsP2 consensus
GSVETPRGLIKVTSY[A/D]GEDKIGSYAVLSPQAVLKSEKLSCIHPLAEQVIVITHSGRKGRY
AVEPYHGKVVVPEGHAIPVQDFQALSESATIVYNEREFVNRYLHHIATHGGALNTDEEYY
KTVKPSEHDGEYLYDIDRKQCVKKELVTGLGLTGELVDPPFHEFAYESLRTRPAAPYQVP
TIGVYGVPGSGKSGIIKSAVIKKDLVVSAKKENCAEIIRDVKKMKGLDVNARTVDSVLIN
GCKHPVETLYIDEAFACHAGTLRALIAIIRPKKAVLCGDPKQCGFENMMCLKVHENHEIC
TQVFHKSISRRCTKSVISVVSTLFYDK[K/R]MRTTNPKETKI[V/E]IDTTGSTKPKQDDLILTCER
GWVKQLQIDYKGNEIMTAAASQGLTRKGVYAVRYKVNENPLYAPTSEHVNVLLTRTEDRI
VWKTLAGDPWIKTLTAKYPGNFTATIEEWQAEHDAIMRHILERPDPTDVFQNKANVCWAK
ALVPVLKTAGIDMTTEQWNTVDYFETDKAHSAEIVLNQLCVRFFGLDLDSGLFSAPTVPL
SIRNNHWDNSPSPNMYGLNKEVVRQLSRRYPQLPRAVATGRVYDMNTGAAATGTLRNYDP
RINLVPVNRRLPHALVLHHNEHPQSDESSFVSKLKGRTVLVVGEKLSVPGK[M/K]VDWLSD[R/Q]P
EATFRARLDIGIPGDVPKYDI[I/V]F[V/I]NVRTPYKYHHYQQCEDHAIKLSMLTKKACLHLNPGG
TCVSIGYGYADRASESIIGAIARQFKFSRVCKPKSS[L/H]EETEVLFVFIGYDRKARTHN[P/S]YK
LSSTLINIYTGSRLHEAGC
SEQ ID NO: 13
>VEEV; SERNA0
GSVETPRGLIKVTSYAGEDKIGSYAVLSPQAVLKSEKLSCIHPLAEQVIVITHSGRKGRYAVEPYHGKVV
VPEGHAIPVQDFQALSESATIVYNEREFVNRYLHHIATHGGALNTDEEYYKTVKPSEHDGEYLYDIDRKQ
CVKKELVTGLGLTGELVDPPFHEFAYESLRTRPAAPYQVPTIGVYGVPGSGKSGIIKSAVTKKDLVVSAK
KENCAEIIRDVKKMKGLDVNARTVDSVLINGCKHPVETLYIDEAFACHAGTLRALIAIIRPKKAVLCGDP
KQCGFFNMMCLKVHENHEICTQVFHKSISRRCTKSVTSVVSTLFYDKKMRTTNPKETKIVIDTTGSTKPK
QDDLILTCFRGWVKQLQIDYKGNEIMTAAASQGLTRKGVYAVRYKVNENPLYAPTSEHVNVLLTRTEDRI
VWKTLAGDPWIKTLTAKYPGNFTATIEEWQAEHDAIMRHILERPDPTDVFQNKANVCWAKALVPVLKTAG
IDMTTEQWNTVDYFETDKAHSAEIVLNQLCVRFFGLDLDSGLFSAPTVPLSIRNNHWDNSPSPNMYGINK
EVVRQLSRRYPQLPRAVATGRVYDMNTGTLRNYDPRINLVPVNRRLPHALVLHHNEHPQSDESSFVSKLK
GRTVLVVGEKLSVPGKMVDWLSDRPEATFRARLDLGIPGDVPKYDIIFVNVRTPYKYHHYQQCEDHAIKL
SMLTKKACLHLNPGGTCVSIGYGYADRASESIIGAIARQFKFSRVCKPKSSLEETEVLFVFIGYDRKART
HNPYKLSSTLINIYTGSRLHEAGC
SEQ ID NO: 14
>ARTIFICAL PROTEIN: TS INSERTION
TGAAA

What is claimed is:

1. A composition for stimulating an immune response against a cancer antigen in a mammalian subject, comprising an excipient, and a temperature-sensitive self-replicating RNA comprising an open reading frame (ORF) encoding a fusion protein, and an Alphavirus replicon lacking a viral structural protein coding region, wherein the ORF comprises from 5′ to 3′:
(i) a nucleotide sequence encoding a mammalian signal peptide; and
(ii) a nucleotide sequence encoding a cancer antigen,
wherein the temperature-sensitive self-replicating RNA is capable of expressing the fusion protein at a permissive temperature but not at a non-permissive temperature.

2. The composition of claim 1, wherein the cancer antigen comprises a tumor-associated antigen (TAA).

3. The comprises of claim 2, wherein the TAA comprises a WT1 antigen, a NY-ESO-1 antigen, a MAGEA3 antigen, a BIRC5 (SURVIVIN) antigen, a PRAME antigen, or a combination thereof.

4. The composition of claim 2, wherein the TAA comprises a WT1 antigen.

5. The composition of claim 4, wherein the amino acid sequence of the WT1 antigen comprises SEQ ID NO:2, or the amino acid sequence at least 95%, 96%, 97%, 98% or 99% identical to SEQ ID NO:2.

6. The composition of claim 2, wherein the TAA is a TAA fusion protein comprising a WT1 antigen, a NY-ESO-1 antigen, a MAGEA3 antigen, a BIRC5 antigen, and a PRAME antigen.

7. The composition of claim 6, wherein the amino acid sequence of the TAA fusion protein comprises SEQ ID NO:7, or the amino acid sequence at least 95%, 96%, 97%, 98% or 99% identical to SEQ ID NO:7.

8. The composition of claim 2, wherein the TAA comprises a BIRC5 antigen.

9. The composition of claim 8, wherein the amino acid sequence of the BIRC5 antigen comprises SEQ ID NO:3, or the amino acid sequence at least 95%, 96%, 97%, 98% or 99% identical to SEQ ID NO:3.

10. The composition of claim 2, wherein the TAA comprises a NY-ESO-1 antigen.

11. The composition of claim 10, wherein the amino acid sequence of the NY-ESO-1 antigen comprises SEQ ID NO:4, or the amino acid sequence at least 95%, 96%, 97%, 98% or 99% identical to SEQ ID NO:4.

12. The composition of claim 2, wherein the TAA comprises a MAGEA3 antigen.

13. The composition of claim 12, wherein the amino acid sequence of the MAGEA3 antigen comprises SEQ ID NO:5, or the amino acid sequence at least 95%, 96%, 97%, 98% or 99% identical to SEQ ID NO:5.

14. The composition of claim 2, wherein the TAA comprises a PRAME antigen.

15. The composition of claim 14, wherein the amino acid sequence of the PRAME antigen comprises SEQ ID NO:6, or the amino acid sequence at least 95%, 96%, 97%, 98% or 99% identical to SEQ ID NO:6.

16. The composition of claim 1, wherein the cancer antigen comprises a neoantigen.

17. The composition of any one of claims 1-16, wherein the mammalian signal peptide is a signal peptide of a surface protein expressed in mammalian antigen presenting cells.

18. The composition of claim 17, wherein the mammalian signal peptide is a CDS signal peptide and the amino acid sequence of the CDS signal peptide comprises SEQ ID NO:1, or the amino acid sequence at least 90% or 95% identical to SEQ ID NO:1.

19. The composition of any one of claims 1-18, wherein the Alphavirus is selected from the group consisting of a Venezuelan equine encephalitis virus, a Sindbis virus, and a Semliki Forrest virus.

20. The composition of claim 19, wherein the Alphavirus is a Venezuelan equine encephalitis virus.

21. The composition of any one of claims 1-20, wherein the Alphavirus replicon comprises a nonstructural protein coding region with an insertion of 12-18 nucleotides resulting in expression of a nonstructural Protein 2 (nsP2) comprising from 4 to 6 additional amino acids between beta sheet 5 and beta sheet 6 of the nsP2.

22. The composition of claim 21, wherein the additional amino acids comprise the sequence of SEQ ID NO:14 (TGAAA).

23. The composition of claim 22, wherein the amino acid sequence of the nsP2 comprises SEQ ID NO:12.

24. The composition of claim 23, wherein the amino acid sequence of the nsP2 comprises one sequence selected from the group consisting of SEQ ID NO:9, SEQ ID NO:10, and SEQ ID NO: 11.

25. The composition of claim 24, wherein the amino acid sequence of the nsP2 comprises SEQ ID NO:11.

26. The composition of any one of claim 1-25, wherein the permissive temperature is from 30° C. to 36° C., or 31° C. to 35° C., or 32° C. to 34° C., or 33° C.±0.5° C., and the non-permissive temperature is 37° C. #0.5° C., optionally wherein the permissive temperature is from 31° C. to 35° C. and the non-permissive temperature is at least 37° C. #0.5° C.

27. The composition of any one of claim 1-26, wherein the composition does not comprise lipid nanoparticles.

28. The composition of any one of embodiments 1-27, wherein the composition further comprises chitosan.

29. A method for stimulating an immune response against a cancer antigen in a mammalian subject, comprising administering the composition of any one of claims 1-28 to a mammalian subject so as to stimulate an immune response against the cancer antigen in the mammalian subject.

30. The method of claim 29, wherein the composition is administered intradermally.

31. The method of claim 29 or claim 30, wherein the immune response comprises a cellular immune response reactive with mammalian cells expressing the cancer antigen.

32. The method of claim 31, wherein the cellular immune response comprises one or both of a cancer antigen-specific cytotoxic T lymphocyte response and a cancer antigen-specific helper T lymphocyte response.

33. The method of claim 32, wherein the immune response further comprises a humoral immune response reactive with the cancer antigen.

34. The method of any one of claims 29-33, wherein the mammalian subject is a human subject.

35. A kit comprising:
(i) the composition of any one of claims 1-28; and
(ii) a device for intradermal delivery of the composition to a mammalian subject.

36. The kit of claim 35, wherein the device comprises a syringe and a needle.
